(19) United States
(12) Patent Application Publication
(10) Pub. No.: US 2012/0065441 A1
(43) Pub. Date: Mar. 15, 2012

(54) DEVICE FOR DISTRIBUTING A POLYPHASE MIXTURE COMPRISING A JET BREAKER TRAY PERFORATED WITH DIFFERENT TYPES OF HOLES

(75) Inventors: Frederic Augier, Saint Symphorien D Ozon (FR); Frederic Bazer-Bachi, Saint Genis-Laval (FR); Christophe Boyer, Charly (FR); Emilie Gagniere, Villeurbanne (FR)

(73) Assignee: IFP Energies nouvelles, Rueil-Malmaison Cedex (FR)

(21) Appl. No.: 13/223,690

(22) Filed: Sep. 1, 2011

(30) Foreign Application Priority Data
Sep. 3, 2010 (FR) 10/03.531
Sep. 3, 2010 (FR) 10/03.532
Oct. 14, 2010 (FR) 10/04.044

(51) Int. Cl. C07C 5/00; B01J 19/00

(52) U.S. Cl. 585/264; 422/224

(57) ABSTRACT

The present invention concerns a device for distributing a polyphase mixture constituted by at least one gas phase and at least one liquid phase, said mixture being in downflow mode passing through at least one bed of solid particles, and said device comprising at least one tray (1) located above a bed of solid particles, a plurality of mixing channels (2) for the liquid and gas phases, a dispersive system of the jet breaker tray type (3) with holes provided with flanges (36) over at least a portion of its perimeter, disposed beneath the mixing channels (2) and above the bed of solid particles, said distribution device being characterized in that the dispersive system (3) comprises at least two types of holes (34, 35).

The present invention relates to the field of the distribution of polyphase fluids in catalytic reactors and more particularly to a device that can be used to optimize the distribution of fluids in catalytic reactors of the fixed bed type, functioning in downflow mode, in applications of the gas oil hydrotreatment type and all hydrogenation operations functioning in gas-liquid trickle flow mode.

In particular, the present invention is applicable to the field of gas/liquid distributors such as, for example, those employed to carry out hydrocracking, hydrotreatment, hydrodesulphurization, hydrodemetallization, hydrodenitrogenation, selective or total hydrogenation, the selective hydrogenation of steam cracked gasoline, the hydrogenation of aromatic compounds in aliphatic and/or naphthenic cuts, and the hydrogenation of olefins in aromatic cuts.

It is also of application in carrying out other reactions necessitating good mixing of a gas phase and a liquid phase, for example partial or total oxidation reactions, amination reactions, acetyloxidation reactions, ammoxidation reactions and halogenation reactions, in particular chlorination.

In general, then, the distribution device is disposed in a vessel or reactor comprising an inlet for a liquid fluid and an inlet for a gaseous fluid, and containing at least one bed, for example of granular solids.

That device may be disposed:
either at the head of the vessel above the bed;
or at the outlet from one bed with a supply over the whole section of the vessel, covering the subsequent bed.

In order to improve the distribution of fluids using said devices, one possibility employed in the prior art consists, for example, of using distributor trays comprising a plurality of mixing channels dedicated to the passage of gas and liquid. Those mixing channels may be of various types and are positioned in various configurations over the tray. Such devices have been described in patent applications FR 2 807 676, FR 2 745 202, FR 2 853 260 or US 2007/0241467.

The disadvantage of that type of mixing channel lies in the fact that the flow leaving the mixing channel forms a relatively concentrated two-phase jet, which is problematic since the liquid is not sprinkled over the whole section of the column. In order to overcome that problem, the spacing between the mixing channels is relatively small (generally between 80 and 200 mm), which considerably increases the number of mixing channels and thus increases the overall cost of the distributor tray.

Another solution to improving sprinkling of the bed is to position jet dispersion elements below the mixing channels. Several types of dispersion elements may be used. Insert type elements are often attached to each mixing channel in order to disperse the jet homogenously over a wider angle below the mixing channel, as described in patent applications EP 2 075 056 and US 2010/0019061.

That type of solution is effective, but it requires a certain distance to be maintained between the mixing channel and the top of the bed of particles so that the jet can sprinkle the bed over a wide area. Ideally, this distance must allow the jets formed by neighbouring mixing channels to join up. Further, that solution is fairly costly due to the large number of dispersion elements to be manufactured.

An alternative solution described in patent FR 2 807 673 consists of positioning jet breaker type dispersion elements beneath the tray which are common to several mixing channels (or even common to an entire row). Conventionally, screens or perforated trays are employed. It is important that the various elements used remain discrete so that the gas can circulate freely between the dispersion elements without being constrained to pass through the elements per se. The gas-liquid jet impinges on the screen and is dispersed over its surface before raining down onto the bed of particles.

Patent FR 2 832 075 describes an improvement to that device which consists of adding flanges solely to the perimeter of the screens. Said flanges allow liquid to remain at the screen and not to overflow at the sides. That type of device has a number of advantages:
it is less expensive than inserts and also means that the spacing between the mixing channels can be increased;
the flow is distributed over the column section and thus there is no need for a jet formation distance to be allowed for beneath the screens.

The disadvantage of that type of device is that it is limited to a certain range of operation. In effect, the geometry of the screen is optimized in a certain range of operation, i.e. for gas and liquid velocities such that:
the two-phase jets leaving the risers are sufficiently broken by the screen and are thus distributed over a certain surface area below the riser;
liquid retention is not too great on the screen, and thus the liquid does not overflow at the sides of the screen, and the liquid level does not pose too many problems if the screen is not very flat.

This tends to limit the optimized range of operation of the screens. In general, it is recommended that operations are always carried out far away from the liquid flow rate at which the screens become clogged, i.e. to use a screen wherein the liquid feed cannot exceed a certain critical height (in the range 0.1 to 1 mm) and thus to use relatively open screens (percentage opening in the range 5% to 20%). In contrast, when a low liquid flow rate is applied in the reactors, the screens do not break the two-phase jet properly and the fluid distribution is not optimized. This is particularly true in operations in which the reactor is not used at its maximum capacity (turndown).

This results in poor distribution of liquid in the bed and a reduction in the reaction performance (loss of conversion and/or selectivity). This type of problem is encountered with mixing channels of the riser type, but are also encountered with mixing channels of the vapour lift type, or of the bubble cap type.

Thus, the present invention is aimed at overcoming one or more of the disadvantages of the prior art by proposing a distribution device comprising a jet breaker tray with an improved range of operation as well as to optimize the distribution of the liquid flow irrespective of the fluid velocity.

To this end, the present invention proposes a device for distributing a polyphase mixture constituted by at least one gas phase and at least one liquid phase, said mixture being in downflow mode passing through at least one bed of solid particles, and said device comprising at least one tray located above a bed of solid particles, a plurality of mixing channels for the liquid and gas phases, a dispersive system of the jet breaker tray type with holes provided with flanges over at least a portion of its perimeter, disposed beneath the mixing channels and above the bed of solid particles, said distribution device being characterized in that the dispersive system comprises at least two types of holes.

In one embodiment of the invention, the dispersive system comprises at least one hole with a flange and at least one hole without a flange.

In one embodiment of the invention, the hole with a flange has a height in the range 0.1 to 5 mm and a diameter in the range 2 to 20 mm.

In one embodiment of the invention, the hole without a flange has a diameter in the range 2 to 20 mm.

In one embodiment of the invention, the hole with a flange comprises a cover.

In one embodiment of the invention, the dispersive system comprises at least one row of holes without a flange alternating with at least one row of holes with a flange.

In one embodiment of the invention, the dispersive system comprises at least one row of holes comprising at least one hole without a flange alternating with at least one hole with a flange.

In one embodiment of the invention, the set of holes is disposed in a staggered or in a square or triangular pattern, in each case with a regular or different spacing.

In one embodiment of the invention, the distribution of the holes is different depending on their position with respect to the centre of the reactor.

In one embodiment of the invention, the dispersive system comprises at least one separating element formed by a solid or pierced or porous plate, having the shape of a planar rectangular parallelepiped, positioned perpendicular to the jet breaker tray.

In one embodiment of the invention, the separating element has a height in the range 20% to 100% that of the flange of the jet breaker tray and closes in the range 40% to 100% of the cross section of the jet breaker tray.

In one embodiment of the invention, the separating element is positioned below a mixing channel or offset with respect to said mixing channel in order to be positioned between two mixing channels.

In one embodiment of the invention, the mixing channels are risers or vapour lifts or bubble caps.

The invention also concerns the use of a device as described above in a reactor adapted for hydrotreatment or hydrogenation or oxidation.

Other characteristics and advantages of the invention will be better understood and will become apparent from the following description, made with reference to the accompanying drawings and given by way of example, in which:

### BRIEF DESCRIPTION OF DRAWINGS

FIG. 1 is a diagrammatic profile view of the distribution device of the invention;

FIG. 2 is a diagrammatic perspective view of the distribution device of the invention;

FIG. 3 is a diagrammatic perspective view of a variation of the distribution device of the invention;

FIG. 4 is a diagrammatic perspective view of another variation of the distribution device of the invention;

FIG. 5 is a diagrammatic perspective view of another variation of the distribution device of the invention;

FIG. 6 is a diagrammatic top view of a variation of the distribution device of the invention;

FIG. 7 is a diagrammatic side view of an example of the use of a distribution device of the invention;

FIG. 8 is a graph representing the height of the liquid level as a function of liquid space velocity.

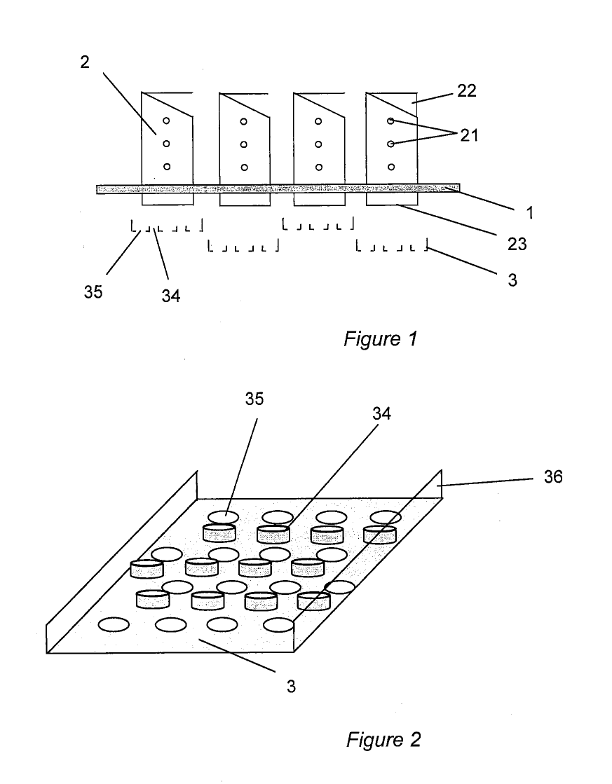

As can be seen in FIGS. 1 and 2, the device of the present invention may comprise a plurality of mixing channels such as risers 2 having at least one upper section of flow 22, for example a taper, at its upper portion, and a lower section of flow 23. These mixing channels may comprise a plurality of holes 21 allowing the passage of liquid. Beneath the tray 1, a jet breaker tray type dispersive system 3 receives the polyphase mixture formed in the mixing channels 2. The height of the mixing channels is usually in the range 100 to 500 millimetres (mm), preferably in the range 200 to 400 mm.

In one embodiment of the invention, the mixing channels may also be vapour lifts. The vapour lift type device (described in patents U.S. Pat. No. 7,600,742 and U.S. Pat. No. 5,942,162) is constituted by tubes forming an M-shaped circuit for circulation of fluid. The term “lift” is used because the vapour initially rises in the device via the outer tubes and then descends via the central tube.

In another embodiment of the invention, the mixing channels may also be bubble caps.

The mixing channel tubes project beyond the tray 1 by a height which normally is between 10 and 200 mm and is often between 25 and 50 mm. Usually, the mixing channel extends below the distributor tray over a length which is less than or equal to the distance between the outlet 23 from a mixing channel and the jet breaker type tray 3.

The portion of the mixing channels 2 disposed above the tray is pierced with holes 21 or slots over its periphery at one or more levels, preferably at least three levels.

The means for ensuring dispersion of the two-phase or polyphase mixture formed in the mixing channel is a jet breaker tray type dispersive system 3 located beneath and in the proximity of the section of flow 23 of the mixing channels. This jet breaker dispersive system is in the form of a jet breaker tray and may either have holes or be porous. The distance between the outlet 23 from the mixing channel and the jet breaker tray 3 usually varies from 5 to 500 mm, usually 10 to 200 mm and preferably 50 to 100 mm. The jet breaker tray is usually constituted by several separated elements disposed at different heights (with respect to the outlet 23 from the mixing channels) but for which the totality of the surfaces covers the section of the reactor. This difference in distance between the various elements of the jet breaker tray and the outlet 23 from the mixing channels means that a free section of flow can be left for the passage of gas. The jet breaker tray 3 is located at a distance from the bed of granular solids in order to conserve the mixture formed inside said mixing channels and leaving said mixing channels via said lower sections of flow until it is distributed into the bed of granular solids. This distance is normally in the range 0 to 500 mm, preferably in the range 1 to 100 mm.

The jet breaker tray may be suspended at the tray 1 or at the lower end of the mixing channels 2.

The jet breaker tray 3 also comprises a flange 36 that can contain the liquid. This flange 36 is disposed over the whole of the jet breaker tray 3, these flanges possibly themselves being porous. The height of the flanges 36 may be in the range 0.2 to 1 times the diameter of the channels, for example in the range 2 to 50 mm. They may themselves have a porosity in the range 0 to 80%. They may or may not be inclined to the vertical, and their inclination is generally in the range −40° to +60°, preferably in the range −30° to +45°, the values of these angles being with respect to the vertical, with positive values corresponding to flanges inclined outwardly of the dispersive system and negative values corresponding to flanges inclined inwardly of the dispersive system. Clearly, when dispersive systems belonging to different horizontal planes are provided with flanges, the distance separating these horizontal planes must be greater than the height of the flanges. The flanges 36 may concern just a portion of the dispersive systems, the other portion not having such flanges. It is often preferable to provide the dispersive systems located on the planes closest to the granular solid with flanges. In certain cases, it may even be advantageous for a given dispersive system to have flanges over only a portion of its perimeter. The precise geometric shape of these flanges may vary; in particular, the upper end of the flanges may be curved inwardly. In the vicinity of the flange of a dispersive system, the porosity of the dispersive system may be zero or identical to the remainder of the surface of the dispersive system 3. The term “in the vicinity of the flange of a dispersive system” means the zone located at a distance of 30 mm or less from the flange and preferably 20 mm or less from the flange.

One of the functions of said flanges and their near-zero porosity is to retain certain impurities that may be contained in the liquid feed, particularly when it is constituted by heavy hydrocarbons such as cuts with a boiling point of more than 350° C., as is the case with units for the hydrotreatment of heavy gas oil type cuts.

In this case, the zone in the vicinity of the flanges gradually becomes laden with impurities, thereby preventing contamination of the bed of granular solids.

The jet breaker type tray of the invention comprises at least two types of holes or perforations:
holes without a flange 35, at screen level;
holes with a flange 34, or raised holes, which are raised with respect to the screen level and which only allow a flow to pass when the guard liquid (i.e. the level of liquid on the jet breaker tray 3) exceeds a certain height.

The holes without a flange 35 (illustrated in FIGS. 1 and 2) may have a diameter in the range 2 to 20 mm, preferably in the range 2 to 15 mm. The same tray may comprise holes without a flange 35 with different diameters.

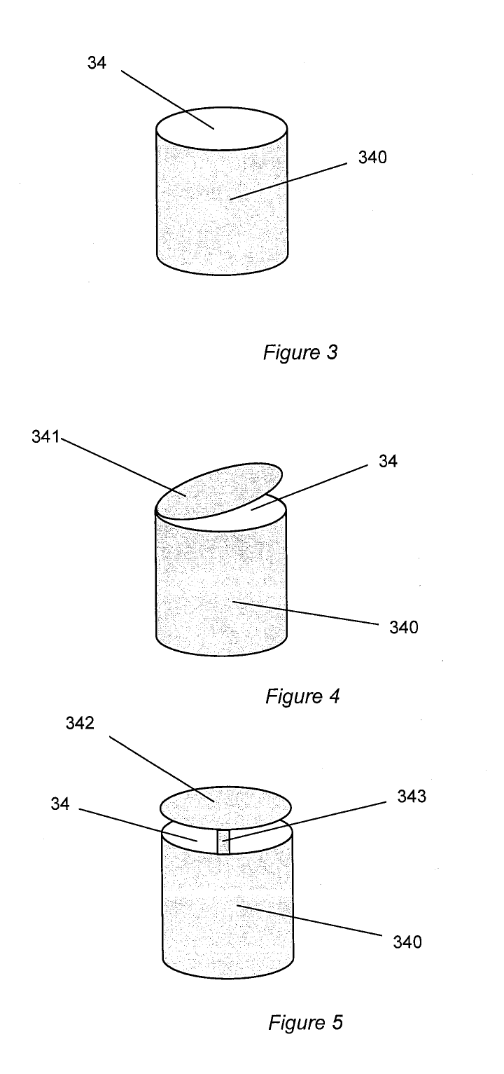

The holes with a flange 34 or risers (illustrated in FIGS. 1 to 5), having the form of a tube, may have a diameter in the range 2 to 20 mm, preferably in the range 2 to 15 mm. The same tray may comprise holes with a flange 34 having different diameters. The height of the flange 340 of the holes with a flange 34 is in the range 0.1 mm to 20 mm, preferably in the range 0.5 to 10 mm. In general, the height of the hole flanges is less than the height of the flange of the tray, preferably in the range 5% to 50% of the height of the tray.

These holes with a flange 34 may comprise a cover 341, 342 or cap as illustrated in FIGS. 4 and 5. The cover 341 may be attached partially and directly to the flange 340 of the hole 34 (FIG. 4). In this manner, the hole remains partially open. The cover 342 may be attached by means of at least two tabs 343 meaning that the cover can remain at a certain distance above the flange (FIG. 5). In this manner, the hole remains completely open but protected by the cover 342.

The holes with a flange 34 of the same or different height, with the same or different diameter and of the holes without a flange 35 of the same or different diameter may be arranged on the tray in any possible manner, non-limiting examples of which are as follows:
at least one row of holes without a flange 35 alternating with at least one row of holes with a flange 34;
each row of holes may comprise a hole without a flange 35 alternating with a hole with a flange 34;
the set of holes (with and without a flange) may be disposed in a staggered pattern or in any other manner, for example in a square or triangular pattern, in each case with a regular or different spacing;
a larger number of holes without a flange 35 than holes with a flange 34; for example, each row may comprise only holes with a flange 35;
the distribution of holes with a flange 35 may be different depending on their position with respect to the centre of the reactor, in order to accommodate any flexing of the tray;
series of holes with a flange 34 of different heights may alternate with holes without a flange 35;
holes without a flange 35 with different diameters along with holes with a flange 34 with different diameters and heights;
a larger number of holes with a flange 34 than holes without a flange 35, for example each row may comprise only holes without a flange 35.

The choice of the number of holes with (34) or with no (35) flange and their diameter is made so as to preserve a percentage opening of the jet breaker tray 3 which in general is in the range 5% to 25%, preferably in the range 5% to 20%.

A preferred embodiment of the invention employs holes without a flange 35 with the same diameter, or even no perforations, with holes with a flange 34 of a few millimetres (0.5-3 mm), with a cover 341, 342. In fact, for low flow rates, it is important to keep the hole density high in order to distribute the liquid properly using small hole sizes for this purpose. For higher flow rates, it is especially important to distribute the excess liquid flow over the whole section of the reactor but the density at the injection point is less critical and the spacing may be a little larger. The cover is necessary in order to prevent liquid from passing through these holes in the absence of a level of liquid on the jet breaker tray: it means that the flow leaving the riser can be distributed over a large surface area of screen for low liquid space velocities.

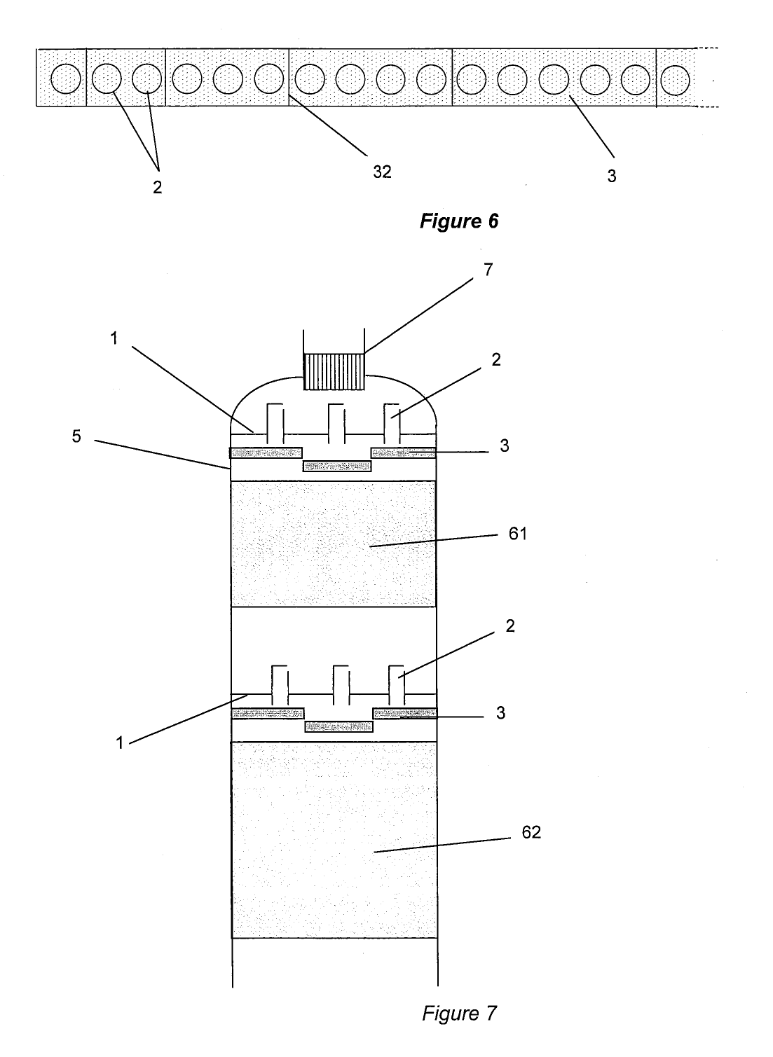

Given that the jet breaker tray of the invention is intended to operate with a level of liquid, the distribution will be sensitive to the jet breaker tray being out of horizontal and it is thus important to be able to insert separating plates perpendicular to the jet breaker tray to limit imbalances in the liquid flow over the jet breaker tray when it is out of horizontal and to prevent the liquid from accumulating under gravity at a single location of the jet breaker and then overflowing in an irregular manner onto the particle bed. In one embodiment of the invention (illustrated in FIG. 6), the jet breaker tray 3 may thus comprise at least one separating element 32, also known as a baffle. The jet breaker tray may comprise one or more separating elements 32. These separating elements may be formed by plates positioned perpendicular to the jet breaker tray. Their height is generally in the range 20% to 100% that of the flange 36 of the jet breaker tray 3, preferably in the range 50% to 90%. They are generally disposed in the plane transverse to the jet breaker tray 3.

In accordance with one embodiment of the invention, the separating elements 32 are orientated in a plane perpendicular to the longest edge of the tray 3.

These separating elements 32 may be disposed in different manners. They may be positioned directly beneath a mixing channel 2 so as to disperse its flow. They may be positioned beneath the mixing channels but offset with respect to the mixing channels so that they are positioned between two mixing channels.

In certain cases, mechanical flexing of the jet breaker trays 3 is not homogenous over the section of the column or reactor. This flexing greatly distorts the tray close to the flange and deforms it less at the centre. It is thus possible to distribute the dispersion elements as a function of their position with respect to the flange 36 of the trays 3. The distribution of the separating elements 32 thus varies as a function of their position on the jet breaker tray 3. As an example, the separating elements 32 may be disposed in an arithmetical manner. In this case, the separating elements 32 are not disposed between the mixing channels 2 in a regular manner but so as to separate a different number of mixing channels 2, for example every 1, 2, 3, 4 etc mixing channels 2. They may also be disposed in greater numbers at the ends of the jet breaker tray 3, for example for every mixing channel 2 in these zones, and every 2, 3, 4 etc mixing channels 2 in the middle. These examples are not in any way limiting and any position for the separating elements 32 may be envisaged. As an example, the separating elements 32 may be disposed every 1 to 10 mixing channels, preferably every 1 to 5 mixing channels.

These separating elements 32 may be in the shape of a planar rectangle or any other shapes adapted to the jet breaker tray 3 used. The separating elements 32 may be solid, pierced or porous in order to partially or completely seal off the cross section of the jet breaker tray 3. Irrespective of their shape, the separating elements close off in the range 40% to 100% of the cross section of the tray, preferably in the range 50% to 100%.

The separating elements 32 are attached to the jet breaker tray and to the flange 36 by conventional attachment means.

The system for attaching the separating elements 23 to the jet breaker tray 3 may also be designed to obstruct a portion of the jet breaker tray 3. The attachment system may, for example, comprise fixing means comprising means for obstructing said type of plate which will plug one or more holes without a flange 35 of the jet breaker tray 3. The plate may be round, oval, square or rectangular in shape, or it may have a different shape. The shape and size of the plate is selected as a function of the number of holes or the surface area of the jet breaker tray 3 to be obstructed. Such a plate may, for example, obstruct one or more holes. The plate may be welded or fixed to the jet breaker tray 3 with an attachment system of the screw and bolt type, or any other attachment system suitable for the device of the invention.

FIG. 7 illustrates a simplified implementation of the distribution device of the invention. The description given below by way of an example of an application concerns a distribution system used in a reactor operating in downflow mode adapted for hydrotreatment.

The reactor comprises a vessel 5 comprising a pre-distributor 7 in its upper portion or reactor head. The mixture distributed by the pre-distributor 7 flows in downflow mode to the distributor tray 1 which is located above a first bed of granular solids 61 or catalytic bed. The tray comprises a plurality of mixing channels 2 opening onto the dispersive jet breaker type tray 3. After its passage through the bed of granular solids 61, the two-phase mixture is re-distributed directly onto a second bed of granular solids 62 after having passed through a second device in accordance with the present invention.

The following examples illustrate the present invention

### EXAMPLES

The following examples illustrate the present invention. The calculations show the change in liquid level on the jet breaker tray as a function of the liquid space velocity (vₛₗ). For proper operation of the jet breaker, it is recommended to use a screen on which the liquid height cannot exceed a certain critical height (in the range 0.1 to 1 mm).

In this height range, the jet breaker may be considered to be more flexible that corresponding to a wide range of V_{A}.

All of the calculations were carried out at a superficial gas flow rate, v_{sg}, of 10 cm/s and at a pressure of 10 MPa.

### Example 1

### Comparative

Use of a jet breaker type tray 3 with holes without a flange 35. The holes were 8 mm in diameter in a 36 mm triangular pattern.

### Example 2

### Comparative

Use of a jet breaker type tray 3 with holes without a flange 35. The holes were 8 mm in diameter in a 22.5 mm triangular pattern.

### Example 3

### In Accordance with the Invention

Use of a jet breaker type tray 3 with holes without a flange 35 and with holes with a flange 34. The holes were 9 mm in diameter in a 25 mm triangular pattern. The holes with a flange were 0.5 mm high. The holes were arranged on the tray in rows of holes without a flange 35 alternating with a hole with flanges 34 (50% of each).

### CONCLUSION

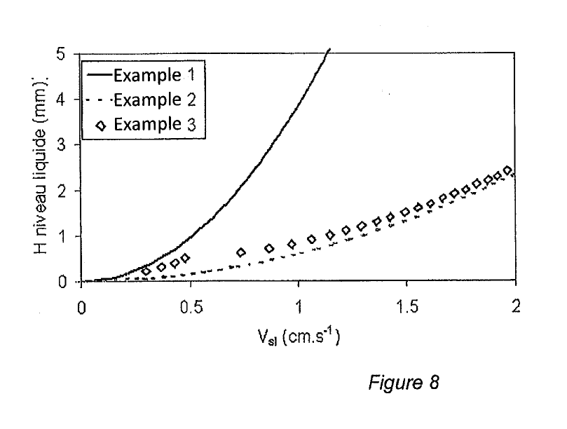

The results for each of the examples are shown in FIG. 8 which represents the height of the liquid level (H, liquid level in mm) as a function of the liquid space velocity (vₛₗ, in cm/s). For the recommended range of liquid height on the screen (in the range 0.1 to 1 mm), the ranges of operation were as follows for the various jet breaker type trays 3:
Example 1: in the range 0.15 to 0.5 cm/s;
Example 2: in the range 0.4 to 1.3 cm/s;
Example 3: in the range 0.2 to 1.2 cm/s.

The range of operation of the jet breaker with two types of holes was thus broader, with the screen operating with a wider range of flow rates.

The present invention is not limited to the details given above; other specific types of embodiments are permitted without going beyond the scope of application of the invention. As a consequence, the present embodiments should be considered to be by way of illustration and may be modified without, however, going beyond the scope as defined in the claims.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding FR application Ser. No. 10/03,532, filed Sep. 3, 2010, FR application Ser. No. 10/03.531, filed Sep. 3, 2010 and FR application Ser. No. 10/04.044, filed Oct. 14, 2010, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A device for distributing a polyphase mixture constituted by at least one gas phase and at least one liquid phase, said mixture being in downflow mode passing through at least one bed of solid particles, and said device comprising at least one tray (1) located above a bed of solid particles, a plurality of mixing channels (2) for the liquid and gas phases, a dispersive system of the jet breaker tray type (3) with holes provided with flanges (36) over at least a portion of its perimeter, disposed beneath the mixing channels (2) and above the bed of solid particles, said distribution device being characterized in that the dispersive system (3) comprises at least two types of holes (34, 35).

2. A device according to claim 1, in which the dispersive system (3) comprises at least one hole with a flange (34) and at least one hole without a flange (35).

3. A device according to claim 2, in which the hole with a flange (34) has a height in the range 0.1 to 20 mm and a diameter in the range 2 to 20 mm.

4. A device according to claim 2, in which the hole without a flange (35) has a diameter in the range 2 to 20 mm.

5. A device according to claim 2, in which the hole with a flange (34) comprises a cover (341, 342).

6. A device according to claim 2, in which the dispersive system (3) comprises at least one row of holes without a flange (35) alternating with at least one row of holes with a flange (34).

7. A device according to claim 2, in which the dispersive system (3) comprises at least one row of holes comprising at least one hole without a flange (35) alternating with at least one hole with a flange (34).

8. A device according to claim 2, in which the set of holes is disposed in a staggered or in a square or triangular pattern, in each case with a regular or different spacing.

9. A device according to claim 2, in which the distribution of the holes is different depending on their position with respect to the centre of the reactor.

10. A device according to claim 2, in which the dispersive system (3) comprises at least one separating element (32) formed by a solid or pierced or porous plate having the shape of a planar rectangular parallelepiped positioned perpendicular to the jet breaker tray (3).

11. A device according to claim 10, in which the separating element (32) has a height in the range 20% to 100% that of the flange (36) of the jet breaker tray (3) and closes in the range 40% to 100% of the cross section of the jet breaker tray (3).

12. A device according to claim 10, characterized in that the separating element (32) is positioned below a mixing channel (2) or offset with respect to said mixing channel (2) so that it is positioned between two mixing channels (2).

13. A device according to claim 1, characterized in that the mixing channels are risers or vapour lifts or bubble caps.

14. A method for hydrotreatment or hydrogenation or oxidation comprising subjecting a feed to reaction conditions in a device according to claim 1.
